(19) United States
(12) Patent Application Publication
(10) Pub. No.: US 2025/0236530 A1
(43) Pub. Date: Jul. 24, 2025

(54) ACCELERATED TOOTH REMINERALIZATION TECHNOLOGY

(71) Applicant: Creighton University, Omaha, NE (US)

(72) Inventors: Stephen M. Gross, Omaha, NE (US); Mark A. Latta, Omaha, NE (US); William A. McHale, Collegeville, PA (US)

(21) Appl. No.: 18/853,560

(22) PCT Filed: Apr. 13, 2023
(86) PCT No.: PCT/US2023/018410

§ 371 Date: Oct. 2, 2024

Related U.S. Application Data
(60) Provisional application No. 63/363,054, filed on Apr. 15, 2022.

(51) Int. Cl. C01D 3/02; A61K 8/21; A61Q 11/00

(52) U.S. Cl. CPC C01D 3/02; A61K 8/21; A61Q 11/00; C01P 2004/03; C01P 2006/12; C01P 2006/40

(57) ABSTRACT

A sodium fluoride hopper crystal formed by a method of manufacture comprising: dissolving a portion of NaF in water to form an NaF solution; forming an antisolvent solution; and adding the NaF solution into the antisolvent solution to precipitate NaF crystals out of the solution.

### CROSS-REFERENCE TO RELATED APPLICATIONS

This application which is a 371 National Phase Entry of International Patent Application No. PCT/US2023/018410 filed on Apr. 13, 2023, which claims the benefit of U.S. Provisional Patent Application No. 63/363,054 filed on Apr. 15, 2022, with the United States Patent and Trademark Office, the contents of which are incorporated herein by reference in their entirety.

### FIELD OF THE INVENTION

The invention is related to processes for forming salt crystals, more particular sodium fluoride salt crystals, compositions comprising the same, and methods for generating rapidly dissolving salt crystals for use in dentifrices and oral care products.

### BACKGROUND OF THE INVENTION

Tooth decay is the number one preventable, noninfectious disease. Primary among this is the formation of caries. Caries formation can predispose the gums to become more susceptible to infection and further infection can lead to systemic infection. Such issues were prevalent throughout history and remain so today. The fluoridation of water has reduced the formation of caries in many countries and so too has the inclusion of fluoride into dentifrices and other topical oral care products. However, poor healthcare remains a significant issue in many underdeveloped areas, where tooth infection and systemic infection remain prevalent. Indeed, in such areas, the use of fluoride varnishes on an occasional basis provides significant improvement and relief to individuals. However, such treatments are often only provided by traveling dental work provided pro bono. In many of these areas, dentifrices are not available, or do not contain fluoride.

However, in many nations, toothpaste and other oral care products utilize fluoride ions, as well as other ions to remineralize tooth surfaces. Teeth are constantly under attack, primarily by acids either ingested into the mouth or by bacteria living on the tooth surfaces. The tooth surfaces are made of enamel, which contains calcium and phosphate. However, these calcium and phosphate ions can be dissolved out of the enamel, weakening, and dissolving this material. The occurrence of this is tooth decay.

While saliva is able to neutralize acids on the tooth surfaces, the constant attack of acids on tooth enamel eventually weakens the enamel. Once the acid is neutralized, the enamel can be remineralized. So long as the remineralization rate is as fast as the demineralization the enamel is protected and dental caries are prevented. However, if the rate of remineralization is slower than the demineralization rate, caries are formed.

The presence of fluoride in the mouth encourages remineralization and also helps to form stronger enamel that is more resistant to future demineralization. Fluoride ions also have an antimicrobial effect and thus fluoride can help prevent and mitigate periodontal disease. However, certain studies have also indicated that increased quantities of fluoride, for example, as provided in drinking water sources, may be causing damage in some cases. Indeed, in certain countries, the presence of fluoride and concentration of the same is closely monitored. For example, the ingestion of high levels of fluoride is potentially disadvantageous, and furthermore, inclusion in drinking water leads to downstream pollution.

Applicant has created methods of manufacture and a novel fluoride salt structure that dramatically increases the rate of dissolution of sodium fluoride salt crystals as compared to ordinary fluoride salt crystals. Such increase in the rate of dissolution has numerous possible impacts including, reducing the quantity of fluoride salts necessary to achieve a comparable therapeutic effect, thereby allowing for lower total quantities of fluoride, increasing the efficacy to ordinary care, because of the rapid dissolution of the materials, improving antibacterial and antimicrobial impacts of the fluoride ion, improving tooth desensitization treatments, and improvements related to caries treatment and prevention. These are explored through the new method of manufacture of the fluoride salt crystal, the fluoride salt crystal itself, uses thereof, and in products using the same.

### SUMMARY OF THE INVENTION

In a preferred embodiment, a method of manufacture of a high surface area (HSA) sodium fluoride (NaF) crystal comprising: (a) dissolving a portion of NaF in water to form an NaF solution; (b) forming an antisolvent solution; and (c) adding the NaF solution into the antisolvent solution to precipitate NaF crystals out of the NaF solution.

In a further embodiment, the method further comprising stirring the antisolvent solution while adding the NaF solution.

In a further embodiment, the method wherein the portion of NaF in water is between 0.8 M and 0.95 M NaF.

In a further embodiment, the method wherein the antisolvent solution comprises an impurity of water.

In a further embodiment, the method wherein the antisolvent solution is ethanol and water. In a further embodiment, the method wherein the antisolvent solution is between 90% ethanol and 85% ethanol.

In a further embodiment, the method wherein the antisolvent to NaF solution is provided at a ratio of 5:1 to 15:1. In a further embodiment, the method wherein the NaF solution is added into the antisolvent solution at a ratio of 10:1 to 15:1. In a further embodiment, wherein the NaF is added dropwise into the antisolvent at room temperature.

In a further embodiment, the method further comprising a drying step comprising removing the antisolvent solution. In a preferred embodiment, wherein the antisolvent solution is vacuum dried. In a further embodiment, the method further comprising drying the HSA crystals and solution at room temperature for 24 hours.

In a further embodiment, the method further comprising mixing the NaF and antisolvent for a predetermined amount of time to allow for HSA crystal formation and upon expiration of the predetermined amount of time removing a layer of antisolvent.

In a preferred embodiment, a sodium fluoride salt crystal formed as a hopper crystal (HSA crystal).

In a further embodiment, the sodium fluoride salt crystal formed by a process comprising: (a) dissolving a portion of sodium fluoride into water to form a sodium fluoride solution; (b) forming an antisolvent solution; and (c) adding the sodium fluoride solution into the antisolvent solution at a ratio of between 1:5 and 1:15 to precipitate hopper crystals and stirring or agitating the antisolvent solution during the addition of the NaF solution.

In a further embodiment, the sodium fluoride salt crystal wherein the antisolvent solution comprises an impurity of water.

In a further embodiment, the sodium fluoride salt crystal wherein the antisolvent is ethanol and water. In a further embodiment, the sodium fluoride salt crystal wherein the antisolvent is between 95% ethanol and 80% ethanol. In a further embodiment, the sodium fluoride salt crystal wherein the antisolvent is between 90% ethanol and 85% ethanol.

In a further embodiment, the sodium fluoride salt crystal wherein the sodium fluoride solution is between 0.8 M and 0.95 M and most preferably about 0.9 M.

In a further embodiment, the sodium fluoride salt crystal wherein the hopper crystals are formed by stirring or agitating the antisolvent solution when adding the sodium fluoride solution.

A method of manufacture of a high surface area sodium fluoride crystal comprising: (a) dissolving a portion of NaF in water to form an NaF solution of between 0.8 M and 0.95 M; (b) forming an antisolvent solution comprising between 85 and 90% ethanol, with water as an impurity; and (c) adding the NaF solution into the antisolvent solution to precipitate NaF crystals out of the NaF solution with the ratio of antisolvent solution to NaF solution at at least 5:1, and preferably at between 5:1 and 15:1, and stirring or agitating the antisolvent during the addition of the NaF solution.

The high surface area sodium fluoride crystal formed by any of the above methods wherein the crystals are defined by a Hall density of 0.68±0.04 g/cm³, and/or a surface area to mass ratio of 0.22±0.04 m²/g.

In a preferred embodiment, use of a sodium fluoride hopper crystal to treat an oral disease.

In a preferred embodiment, use of a sodium fluoride hopper crystal to form an oral dental product wherein the oral dental product is nonaqueous.

In a preferred embodiment, an oral care product comprising a nonaqueous carrier and a portion of sodium fluoride hopper crystals of the present disclosure. Preferably, the sodium fluoride hopper crystals of the present disclosure are used at less than 1,500 ppm, and most preferably at less than 750 ppm. In preferred embodiments the sodium fluoride concentration is defined at a 5% w/w % concentration, and in certain embodiments, the oral care product having a sodium fluoride concentration at 2.5% w/w %.

In a preferred embodiment, a method of manufacture of a high surface area (HSA) sodium fluoride (NaF) crystal comprising: (a) dissolving a portion of NaF in water to form an NaF solution; (b) forming an antisolvent solution; and (c) adding the NaF solution into the antisolvent solution to precipitate NaF crystals out of the NaF solution.

In a further embodiment, the method further comprising stirring the antisolvent solution while adding the NaF solution. In a further embodiment, the method wherein the NaF solution is between 0.8 M and 0.95 M NaF.

In a further embodiment, the method wherein the antisolvent solution comprises an impurity of water.

In a further embodiment, the method wherein the antisolvent solution is ethanol and water. In a further embodiment, the method wherein the antisolvent solution is between 80% and 95% ethanol and most preferably between 85% and 90% ethanol.

In a further embodiment, the method wherein the ratio of antisolvent solution to NaF solution is 5:1 to 15:1. In a further embodiment, the method wherein the ratio of antisolvent solution to NaF solution is 10:1 to 15:1.

In a further embodiment, the method further comprising a drying step comprising removing the antisolvent solution.

In a further embodiment, the method further comprising mixing the NaF and antisolvent solution for a predetermined amount of time to allow for HSA crystal formation and upon expiration of the predetermined amount of time removing a layer of antisolvent; and wherein the predetermined amount of time is preferably between 1 hour and 24 hours.

In a further embodiment, the method further comprising one or more of the following steps: (a) drying the HSA crystals and solution at room temperature for 24 hours; and/or (b) vacuum drying the HSA crystals; and/or (c) placing a container containing formed crystals into an oil bath at 100° C.

In a preferred embodiment, a high surface area (HSA) sodium fluoride (NaF) salt crystal formed as a hopper crystal made by any preceding method.

In a further embodiment, the HSA NaF salt crystal wherein the HSA NaF salt crystals are defined by a Hall density of 0.68±0.04 g/cm³ and/or a surface area to mass ratio of 0.22±0.04 m²/g.

In a preferred embodiment, use of a sodium fluoride hopper crystal to treat an oral disease.

In a preferred embodiment, use of a sodium fluoride hopper crystal to form an oral dental product wherein the oral dental product is nonaqueous.

In a preferred embodiment, an oral care product comprising a nonaqueous carrier and a portion of sodium fluoride hopper crystals.

In a further embodiment, the product wherein the sodium fluoride hopper crystal is made by any preceding method. In a further embodiment, the product wherein the sodium fluoride hopper crystal comprises a Hall density of 0.68±0.04 g/cm³ and/or a surface area to mass ratio of 0.22±0.04 m²/g.

In a preferred embodiment, an oral care product comprising NaF made by any preceding method wherein the NaF is provided in an oral care product at 2.5 w/w % and provides a release profile of at least 50 ppm/g formulation within 50 minutes.

In a preferred embodiment, a method of manufacture of a high surface area (HSA) sodium fluoride (NaF) crystal comprising: (a) dissolving a portion of NaF in water to form an NaF solution of at least 0.8 M; (b) forming an antisolvent solution comprised of ethanol and water with ethanol at between 80% and 95%; and (c) adding the NaF solution into the antisolvent solution to precipitate NaF crystals out of the NaF solution while agitating the antisolvent solution.

In a further embodiment, the method wherein the NaF is added into the antisolvent solution dropwise.

In a further embodiment, the method wherein step (c) is performed at room temperature.

In a preferred embodiment, a sodium fluoride crystal having a Hall density of 0.68±0.04 g/cm³ and/or a surface area to mass ratio of 0.22±0.04 m²/g which is made by a method comprising the following steps: (a) dissolving a portion of NaF in water to form an NaF solution of at least 0.8 M; (b) forming an antisolvent solution comprised of ethanol and water with ethanol at between 80% and 95%; and (c) adding the NaF solution into the antisolvent solution to precipitate NaF crystals out of the NaF solution while agitating the antisolvent solution.

### BRIEF DESCRIPTION OF THE FIGURES

FIG. 1A (Prior Art) depicts a scanning electron microscope (SEM) image of ordinary commercially available cubic sodium fluoride crystals, as would be understood in the prior art. FIG. 1B depicts a SEM image of an experiment that generated a small portion of high surface area (HSA) sodium fluoride crystals, with two of the HSA crystals being circled in the figure. FIG. 1C depicts an image of the resultant product depicting a high percentage of HSA crystals being formed.

FIGS. 2A-2F depict SEM images from a series of sodium fluoride crystals produced from the addition of 0.9 M NaF added to antisolvent (ratio of antisolvent to NaF was 10:1), while stirring at 1,000 RPM. The results of each of the different figures test different ethanol concentrations, beginning at 100% in FIG. 2A, and through the addition of a percentage of water, yielding the percentages 95%, 90%, 85%, 80%, and 75% ethanol.

FIGS. 3A and 3B depict SEM images from samples, with FIG. 3A depicting a ratio of 1:10 salt solution to antisolvent depicting nearly 90% formation of high surface area crystals and FIG. 3B depicting a ratio of 1:15 salt solution to antisolvent depicting nearly 90% formation of high surface area crystals. FIGS. 3C-3F depict additional SEM images testing the modification of the antisolvent:NaF ratio, when tested at 90% ethanol, while stirring at 1,000 RPM.

FIG. 4A depicts an SEM image related to testing of stirring the sodium salt in antisolvent solution and generating quantitative yields of high surface area crystals by stirring a 1,000 RPM, with the results at 100 RPM being substantially the same. FIGS. 4B, 4C, and 4D show three different SEM images, by testing a 0.9 M NaF solution, added to antisolvent (85% ethanol) at a 5:1 antisolvent:NaF ratio with different levels of stirring during the dropwise addition of the NaF solution.

FIG. 5 depicts the conductivity of two different versions of sodium fluoride, with the commercially available material being a generic crystal form and the high surface area sodium fluoride crystals as made by the present disclosure, with the conductivity measured against time.

FIG. 6A depicts a graph comparing two different precipitation experiments for the high surface area crystals compared to the commercial cubic crystals for conductivity. FIG. 6B provides a further graph testing the same antisolvent ratio, from 1.25:1 to 15:1 using a 90% ethanol solution.

FIG. 7A depicts a graph depicting two different precipitation conditions and comparing the formation of the high surface area crystals to the ordinary commercial cubic crystals for conductivity. FIG. 7B shows reaction conditions for a 10:1 antisolvent:NaF ratio while 1,000 RPM stirring for 95%, 90%, 80%, and 85% ethanol.

FIG. 8A depicts a graph showing conductivity of two different reaction conditions for the high surface area crystals of the present disclosure. FIG. 8B depicts conditions for no stirring, 100 RPM, and 1,000 RPM under ideal conditions of 85% ethanol and a 5:1 ratio of antisolvent:NaF.

FIG. 9A depicts a graph showing conductivity of a high surface area crystal compared to a commercial cubic crystal, each at a 5% w/w release from a fluoride varnish. FIG. 9B depicts a graph showing that HSA crystals have a similar profile for release of sodium fluoride at a 2.5 w/w % formulation as compared to the 5 w/w % commercial product.

FIGS. 10A, 10B, and 10C show different HSA crystals, with FIG. 10A depicting hollow cube type crystals, FIG. 10B depicting a skeletal crystal, and FIG. 10C depicting a magnified version of one skeletal sodium fluoride crystal.

### DETAILED DESCRIPTION OF THE INVENTION

The role of fluoride, calcium, and phosphate ions in remineralizing the tooth structure is well documented. Fluoride also has an antimicrobial effect that can help mitigate periodontal disease. The addition of remineralizing agents to dental products such as dentifrices, whitening gels, varnishes, other topical moieties, restorative materials, cements, adhesives, glazes, endodontic sealers, and other oral care and professional dental products is commonplace. One challenge for creating products capable of action (whether remineralizing or other) on the tooth structure is that these materials need to be in contact with the oral hard or soft tissue for a sufficient amount of time to deliver these therapeutic fluoride agents in the oral environment in a structure that dissolves faster than conventionally manufactured materials. These ions are generally introduced into oral care materials as a salt.

Therefore, these various salt crystals are an important part of many oral care materials, as it is fluoride and calcium salts that are frequently utilized in these materials. Fluoride salts are commonly introduced as sodium fluoride, while calcium salts may include several different materials including calcium chloride, calcium phosphate or others. Salts are a combination of at least two ions, and the salt, when dissolved, releases the positive and negative ions that make up the salt.

Commercially available sodium fluoride (NaF) salt crystals are commonly sold and used as solid cubes or flakes that range in size from 45-600 μm. Molecularly, these cubes organize in a crystalline structure where both the Na⁺ and F⁻ occupy octahedral coordination sites. Due to their large size and morphology, it takes several minutes to fully dissolve a typical sodium fluoride salt crystal in the mouth. While the FDA requires toothpastes to dissolve a given portion of the sodium fluoride within a given time, such test is performed by vortexing the material to increase the dissolution rate, conditions that are not replicated by ordinary brushing. Accordingly, under normal oral care use, a portion of the toothpaste is commonly swallowed while the remaining portion is expectorated. Furthermore, most individuals typically brush from between 10 seconds and 60 seconds, which, as is shown in the figures, leaves a significant portion of the ordinary solid cubic sodium fluoride salt crystals from the toothpaste in an undissolved form.

Thus, a portion of the fluoride salt crystal is swallowed or expectorated, instead of being dissolved into the mouth, with the expectorated portion leading to downstream wastewater pollution. Furthermore, as is detailed in the figures, the ordinary solid cubic sodium fluoride crystal does not reach 90% dissolution in water for several minutes, thus it is not dissolved to this point in ordinary brushing. Thus, a lower amount of the sodium and fluoride ions are actually released to be adjacent to a tooth surface to be utilized for remineralization than would be found with a faster dissolving salt. Thus, the efficiency and efficacy of the fluoride is not maximized.

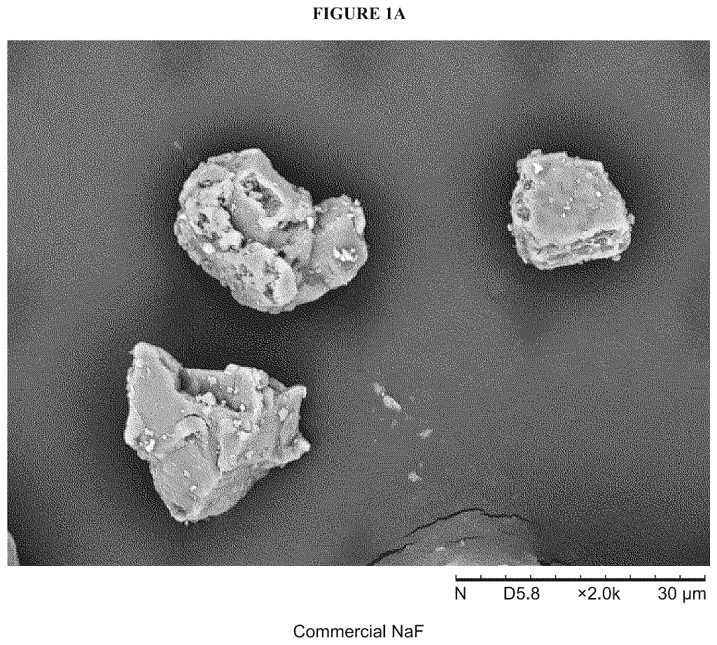

There is a need for a salt with a greater dissolution rate than ordinary commercially available solid cubic sodium fluoride salt crystals, such as those depicted in FIG. 1A. A further benefit would also include a NaF salt that generates a similar effective concentration of fluoride ions in the oral cavity over the time period of tooth brushing, while using lower total amounts of the NaF salt. Such an impact would allow for an equivalent therapeutic level of the fluoride to be reached in the mouth, while using less total mass than existing solid cubic salt crystals. The use of a sodium fluoride salt crystal that dissolves at a faster rate would increase the efficacy of oral care products through a more rapid release of ions into the mouth and to tooth surfaces. Furthermore, it would also be possible to reduce the total quantity of the sodium fluoride salt being utilized in the oral care product while still reaching an equivalent therapeutic level in the mouth and would thus reduce the quantity of the fluoride that is swallowed as well as reduce the downstream pollution impacts of these salts.

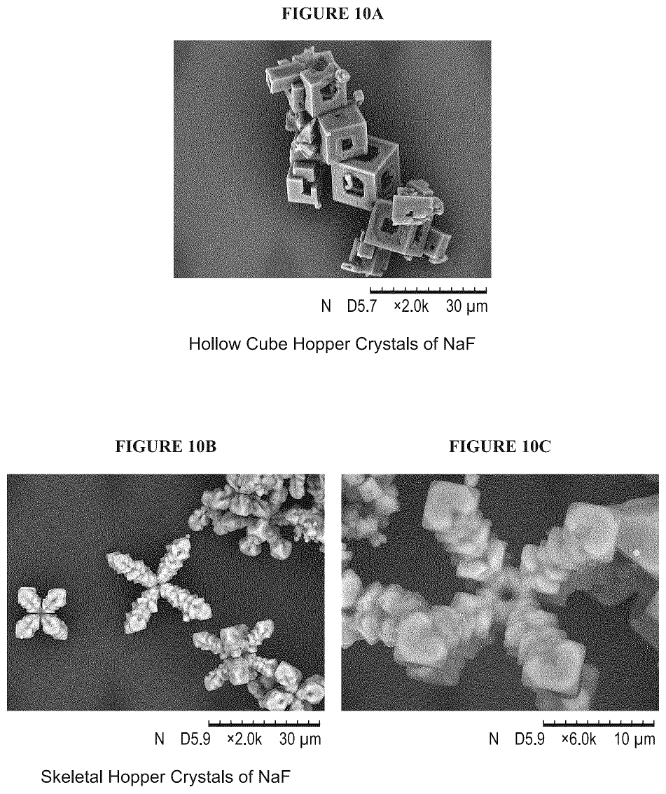

Salt crystal morphology can be altered under certain processes, and applicant has identified a process for generating unique sodium fluoride salt crystal structures through specific concentrations of solutions, use of a diluted antisolvent, and mechanical stirring to optimize the formation of high surface area crystals (“HSA crystals”), which may also be called hopper crystals. These HSA crystals exhibit a higher rate of dissolution than existing cubic sodium fluoride salt crystals when dissolved in aqueous environments, likely due to their different morphology, which exhibits a much higher surface area to mass ratio than conventional cubic salt crystals. Examples of HSA crystals include hollow cube NaF crystals, as depicted in FIG. 10A, as well as skeletal hopper crystals of NaF, as depicted in FIGS. 10B and 10C. These structures show a striking difference to the prior art crystals in FIG. 1A, each having more open structure, and thus a much higher surface area to mass ratio. Accordingly, disclosed are methods of manufacture of HSA crystals and products that contain the same.

The remineralizing agents in this invention are high surface area sodium fluoride salt crystals. These HSA sodium fluoride salt crystals are synthesized with significantly higher surface area to volume ratios than conventionally manufactured solid cubic salt crystals and can therefore create a much higher concentration of ions, upon application of the HSA crystals in a solvent, whether dissolved alone or from a dental material, in less time than commercial solid cubic sodium fluoride salt crystals. The shape and structure of the HSA crystals of the present disclosure are verified by several SEM images of the HSA crystal fluoride salt. These HSA crystals typically exhibit shapes such as a hollow cube (like FIG. 10A) or shaped like a jack (like FIG. 10B) or other shapes that greatly increase the surface area as compared to ordinary solid cubic-shaped crystals. Furthermore, conductivity graphs as a function of time show the greater rates of dissolution of the HSA crystal as compared to a commercial solid cubic sodium fluoride salt.

### Conditions for the Production of Accelerated Remineralizing Agents

The maximum solubility of sodium fluoride in water at room temperature is 0.98 M, therefore working at ˜90% to 99% saturation of the salt in water was targeted. This was done in order to maximize the yield of the materials, without reaching a supersaturated solution. Thus, a 0.8 M to about a 0.95 M solution was utilized, with 0.9 M used in many of the test examples.

Formation of hopper crystals can be achieved through control of the solute concentration gradient with various methods which include crystal growth in the vapor phase, the solution phase, gel systems, and electrocrystallization. It has been determined that concentration gradient in solution is the most important factor in crystal formation, as many crystals can only grow from aqueous solution. A solution is said to be saturated when the maximum amount of solute has been dissolved by the solvent. Precipitation then occurs when solute is added to solution beyond the solvent's ability to solvate the particles. Under certain conditions, regular cubic crystals precipitate from a supersaturated solution. Conditions can be modified however to induce hopper-shaped crystals, including use of an antisolvent or rapid evaporation of the solvent in confinement. Ions in solution form a primary nucleation site at which precipitation of a crystal occurs. Crystal growth occurs rapidly in a supersaturated solution due to the excess of ions available. As ions are being added to the growing structure, solute concentration decreases in certain areas of the bulk solution. Specifically, solute concentration near the center of the crystal is less than the concentration near the outer edges. Growth then becomes limited by how quickly ions can be incorporated. In order for growth to continue at a rapid rate, ions are added to the corners and edges of the crystal more quickly than the face centers. Nucleation and growth of a crystal on the corner or edge of an existing crystal gives rise to the hopper morphology.

In order to create HSA crystals, a 0.9 M solution of sodium fluoride is prepared by dissolving ordinary solid cubic sodium fluoride crystals into water and the solution is then added into an antisolvent. The antisolvent is a substance in which sodium fluoride is nearly insoluble, especially when compared to the solubility of sodium fluoride in water. The antisolvent is preferably miscible with water. Accordingly, as a nonlimiting example, this can include a number of lower chain alcohols, such as C2-C10 alcohols. A common alcohol in this range is ethanol, which is appropriate for its ease of use, its low cost, and its compatibility with human consumption. Applicant tested ethanol, acetonitrile, and acetone as antisolvents. Upon initial testing, while some HSA crystals were formed with acetonitrile, as well as different concentrations of ethanol, ethanol was determined to be an optimized choice for certain tests.

In order to create the HSA crystals, the solution of sodium fluoride is added dropwise into an antisolvent to precipitate the HSA crystals. For example, this can be performed, as follows: in a container an antisolvent for sodium fluoride is added, such as 90% ethanol. Then, a sodium fluoride solution is dripped into the antisolvent to precipitate crystals, preferably while stirring the fluids in the container. The particular reaction conditions, however, are critically important for generating the HSA crystals and for improving yield of the same, instead of forming standard solid cubic crystals.

Three variables significantly affected the outcome of producing HSA NaF crystals. These variables included: (1) the purity of the antisolvent purposefully diluted with water, (2) the antisolvent to salt solution ratio at the end of the experiment, and (3) whether the system was mixed or not during the course of the addition of the salt solution to the antisolvent. Presumptively the initial concentration of sodium fluoride would have an effect, too. However, this was not varied as the largest concentration of product would obviously be desired from a manufacturing perspective. The maximum solubility of sodium fluoride in water at room temperature is 0.98 M, therefore working at ˜90% saturation of the salt in water was targeted, however a solution up to saturation is likely to be effective and even supersaturated may also be effective.

The following experiments were evaluated to generate working conditions for the material and to optimize the production and yield of the HSA crystals. Each of the tests were performed at room temperature.

### Effect of Antisolvent Purity

A number of antisolvents were explored for this experiment, including acetonitrile, acetone, and different concentrations/purity of ethanol. Conceptually, the sodium fluoride produced in this invention would be used in the human body, and thus a material that may impart dangerous residues to the crystals is a poor choice. Thus, while numerous antisolvents would allow for formation of the HSA crystals, early results showed greater concentrations generated with ethanol, and thus the acetonitrile and acetone were discontinued. Therefore, the descriptions that follow will focus on the use of ethanol as an antisolvent.

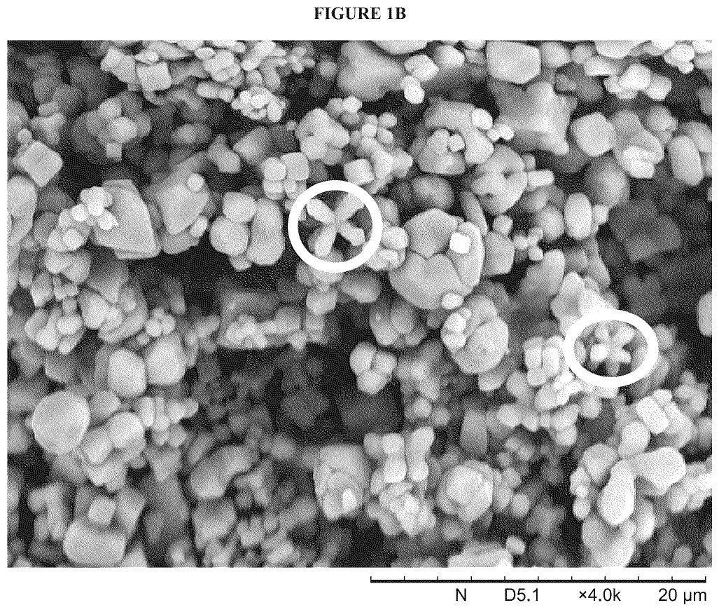

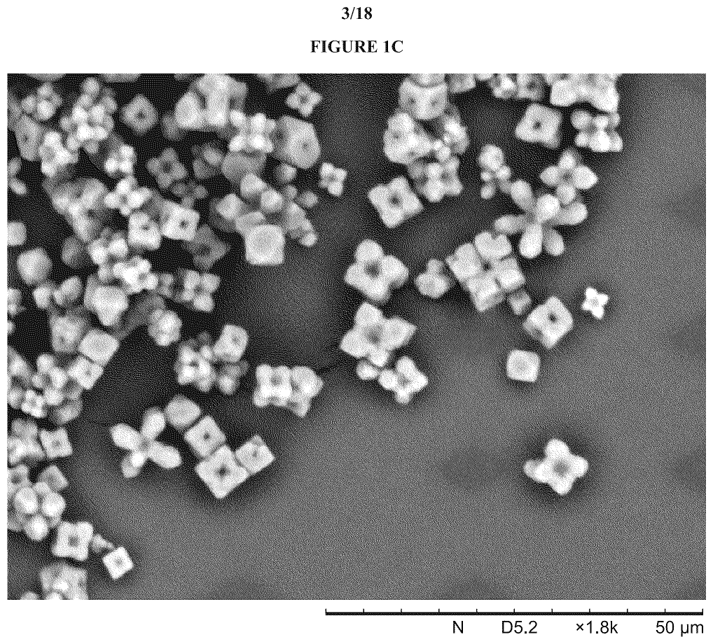

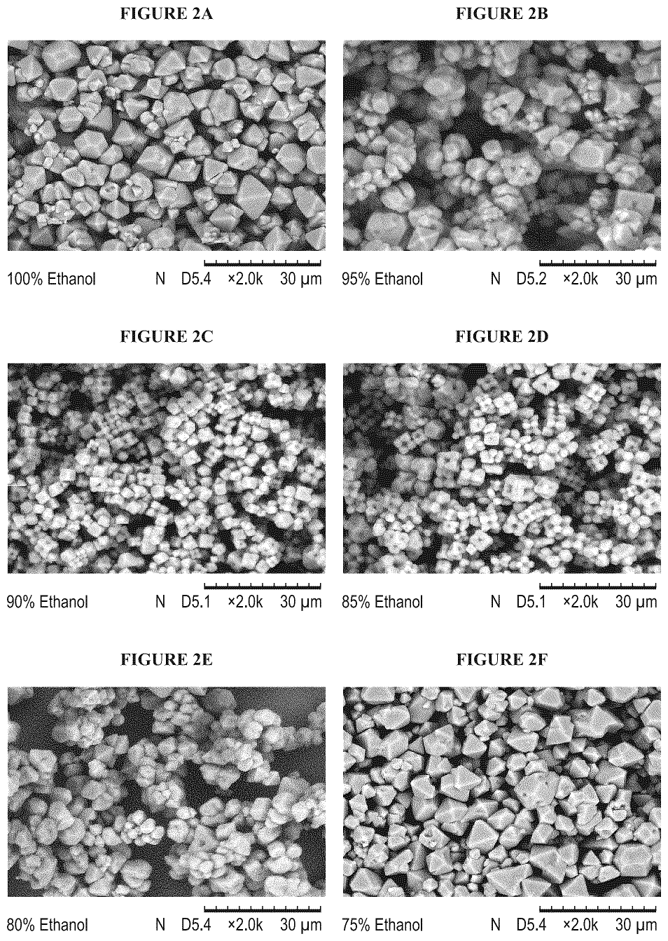

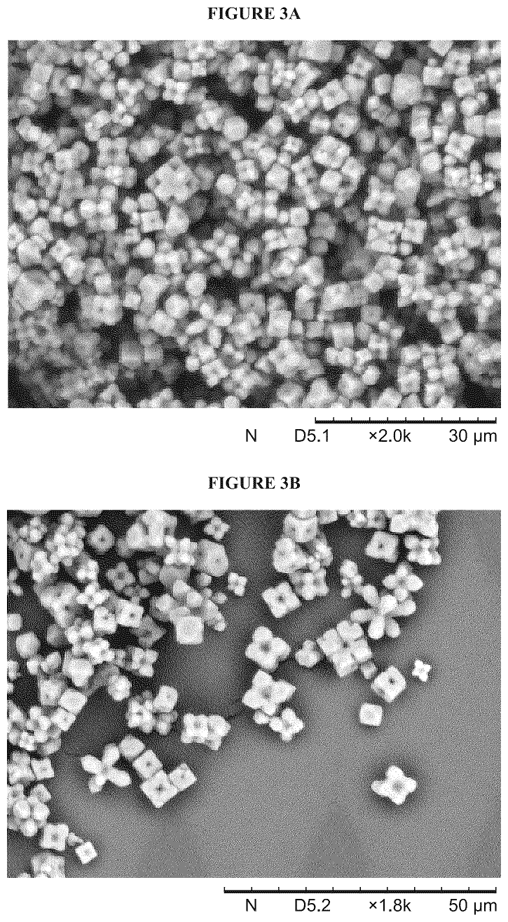

Accordingly, a simple preparation of a sodium fluoride solution is prepared at 0.9 M and added to an antisolvent. Initially the antisolvent was pure ethanol. When 10 mL of 0.9 M NaF was dripped into 100 mL of 100% pure ethanol while stirring at 1,000 RPM, there were no HSA crystals with the higher surface area morphology as seen in the SEM image of FIG. 2A. Indeed, the crystals of FIG. 2A simply show a crystal morphology that mirrors that of ordinary commercially available solid cubic crystals. The same results were obtained at concentrations of both 1.25:1 and 5:1 antisolvent to NaF solution. Therefore, use of 100% ethanol produced no HSA crystals, even when modifying the ratio of antisolvent to the NaF solution.

However, by modifying the percent of ethanol concentration, with water mixed into the ethanol, Applicant was unexpectedly able to generate HSA NaF crystals. The following antisolvent:water mixtures were prepared: 95% ethanol:5% water, 90% ethanol:10% water, 85% ethanol:15% water, 80% ethanol:20% water, and 75% ethanol:25% water. As the purity of the ethanol initially decreased, the presence of some high surface area crystals was observed as seen, as depicted in FIG. 2B. Samples using 95% ethanol and 5% water generated between 12% and 20% of HSA crystals, while using 90% ethanol and 10% water generated between 72% and 84% HSA crystal formation in various tests, as depicted in FIG. 2C.

When the amount of water added to the antisolvent prior to addition of the salt increased to create an antisolvent ratio of 85% ethanol:15% water nearly all of the NaF precipitated as high surface area crystals as seen in the SEM of FIG. 2D. Indeed, results showed the formation of HSA crystals was between 88% and 92% of all crystals.

However, by further increasing the addition of water to the antisolvent, the yield was dramatically reduced. Indeed, at the 80% and 20% tests, the HSA crystals are formed at between 0% and 18%, as seen in the SEM as depicted in FIG. 2E. Finally, at 75% and 25%, no HSA crystals were formed as depicted in FIG. 2F. Therefore, Applicant unexpectedly identified that specific reaction conditions regarding the antisolvent were necessary for the formation of the HSA NaF crystal formation. The reaction conditions are summarized in Table 1, which shows the impacts of the purity of antisolvent on the formation of the high surface area crystals.

TABLE 1

| | | | HSA Crystal |
|---|---|---|---|
| Test No | Percent Water | Percent Ethanol | Formation |
| 1 | 0 | 100 | None |
| 2 | 5 | 95 | Approx. 15% |
| 3 | 10 | 90 | Approx. 75% |
| 4 | 15 | 85 | Nearly 90% |
| 5 | 20 | 80 | Few, approx. 10% |
| 6 | 25 | 75 | None |

Therefore, working conditions are preferably between 5% and 20% of water with 80% to 95% ethanol for the antisolvent. Preferably, the range falls between about 7% and 18% water, and most preferably, between about 8% and 17.5%, or between 8.5% and 17%, with an optimal range of between 9% and 17%, to maximize yield.

It was hypothesized that adding water to another solvent would also increase yield, and it was tested with acetonitrile, at 95% and 90%, though the results revealed between 0% and 20% yield, and only under high agitation. Although some conditions achieved up to 20%-30% hopper crystal morphology, this was not close to the success achieved with ethanol as the antisolvent and was not further explored. Thus, the use of water improved even the acetonitrile solvent, but not as substantially as with ethanol. Therefore, the ethanol and water mixture provided unexpectedly superior rates of HSA formation of the NaF crystals.

### Ratio of Salt Solution to Antisolvent

The ratio of total volume of 0.9 M sodium fluoride salt solution added to the total volume of antisolvent (85% ethanol) was changed over the course of experiments. The volume of salt solution added to antisolvent ranged from a ratio of 1.25 mL of antisolvent:1 mL salt solution; to a ratio of 15 mL of antisolvent:1 mL salt solution. In each experiment, after the aqueous sodium fluoride solution is prepared, it is added to an antisolvent. In each of the tests, 10 mL of 0.9 M NaF was dripped into various volumes of 85% ethanol while stirring at 100 RPM. In the first experiment, 10 mL of 0.9 M sodium fluoride solution was added to 12.5 mL of 85% ethanol with stirring, no HSA NaF crystals with the higher surface area formed.

When 10 mL of 0.9 M sodium fluoride solution was added to 50 mL of 85% ethanol with stirring, the presence of some high surface area crystals was observed. However, when 10 mL of sodium fluoride solution was added to 100 mL of 85% ethanol while stirring at 100 RPM, the yield of HSA crystals was approximately 90%.

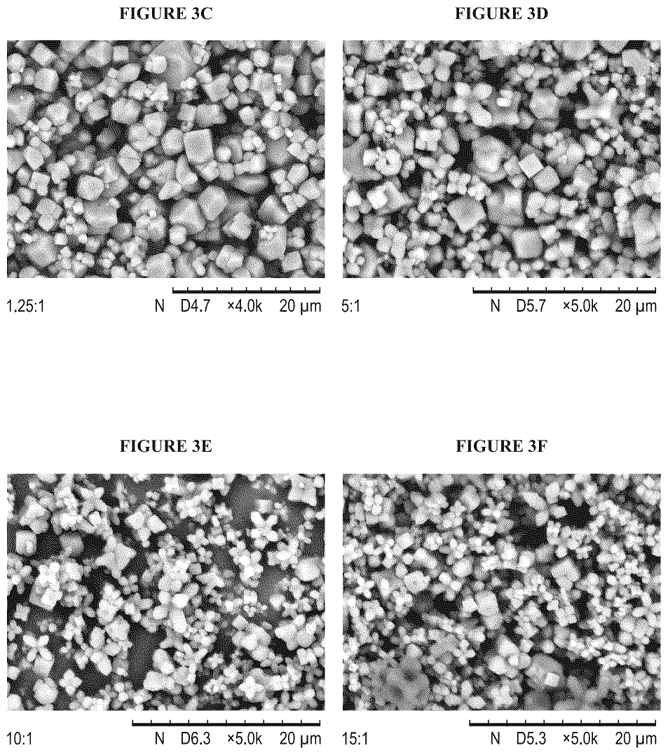

Finally, when 10 mL of sodium fluoride solution was added to 150 mL of 85% ethanol while stirring at 100 RPM, the yield of HSA crystals was also approximately 90%. The HSA crystals generated from the tests performed at 10:1 and 15:1 are depicted in FIGS. 3A and 3B. Practically, there would be no purpose to use a greater ratio of antisolvent as this would increase the cost of manufacturing. Accordingly, the results can be summarized in Table 2 as follows.

TABLE 2

| | Volume | | |
|---|---|---|---|
| Test No | Antisolvent | Volume NaF | Result |
| 6 | 12.5 mL | 10 mL | No crystals |
| 7 | 50 mL | 10 mL | Few crystals |
| 8 | 100 mL | 10 mL | Approx. 90% HSA crystal |
| 9 | 150 mL | 10 mL | Approx. 90% HSA crystal |

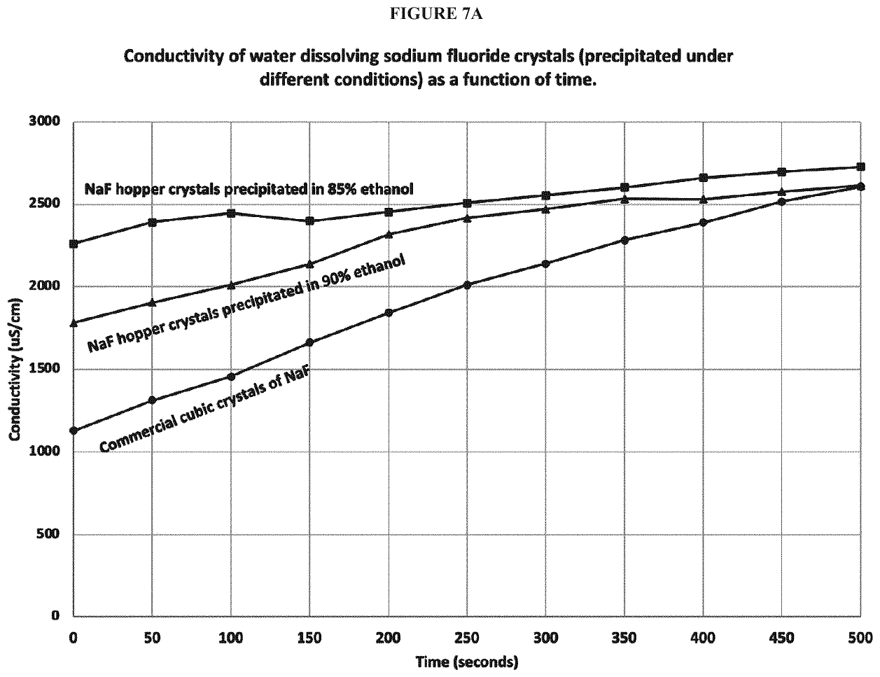

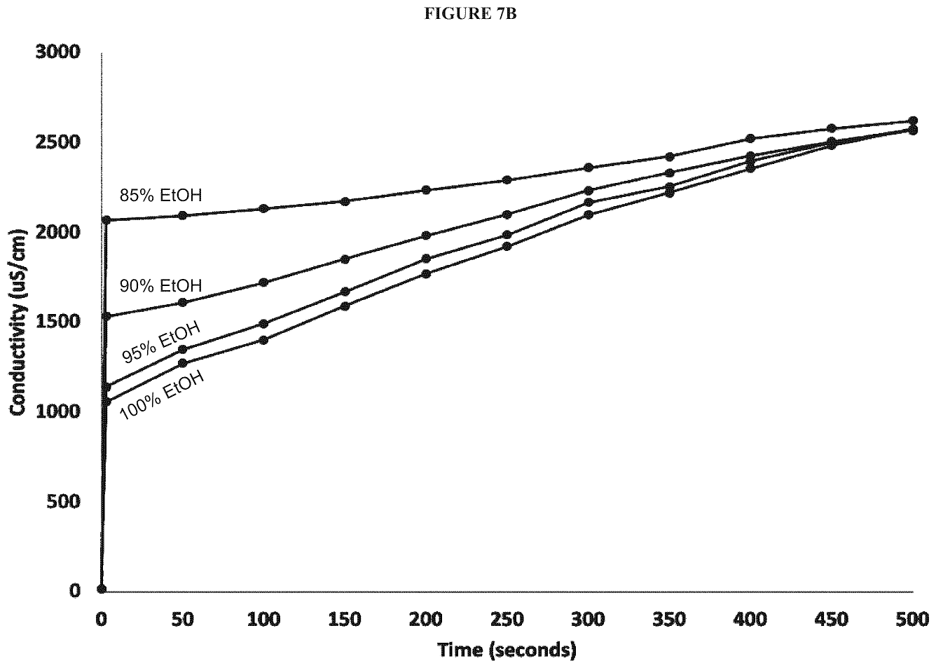

The test was repeated and replaced the 85% ethanol antisolvent for a 90% ethanol antisolvent and stirring at 1,000 RPM. FIGS. 3C, 3D, 3E, and 3F, repeat the tests above with 1.25:1, 5:1, 10:1, and 15:1 ratios tested. The results of that experiment repeat the results as found at the 85% ethanol, with no crystals formed at the 1.25:1 ratio in FIG. 7C, a few crystals formed at the 5:1 ratio in FIG. 7D, and significant HSA NaF crystal formation at the 10:1 and 15:1 ratios as depicted in FIGS. 3D and 3F.

Therefore, when forming the crystals, the ratio of the volume of antisolvent:NaF must be at least 5:1, more preferably at least 8:1, and most preferably 10:1 or higher. In preferred embodiments, the range of antisolvent:salt solution is between 5:1 and 15:1, with the most preferred range of between 8:1 and 17:1, and preferably about 9:1 and 16:1, with a preferred ratio of 10:1 to 15:1. The higher range may be increased, however, at 90% formation of high area salt crystals, a ratio of greater than 15:1 does not appear to create a better yield and would only add cost to the process. Thus, the lowest concentration generating the highest yield is desired, which is between 10:1 and 15:1.

### Requirement for Stirring

The final variable tested for growth of HSA crystals was the effect of stir rate during addition of the sodium fluoride salt solution to the antisolvent. Based upon the prior experiments, an optimized set of parameters was utilized where formation of the HSA crystals was generated at or about 100% formation in order to determine the amount of stirring necessary to generate the HSA crystals.

Stirring is believed to be required because the rate of addition of the ions into the solid is hypothesized to be a limiting step of the crystal morphology. Thus, as detailed below, when no stirring occurred, ordinary solid cubic crystals are formed, while the addition of stirring allows for the formation of the HSA crystals.

In the following experiments, 10 mL of 0.9 M sodium fluoride solution was added to 150 mL of 85% ethanol with either (1) no stirring (the sodium fluoride was “layered” on top of the antisolvent), (2) stirring at 100 revolutions per minute (RPM), or (3) stirring at 1,000 RPM.

In the first experiment where 10 mL of the 0.9 M sodium fluoride salt solution was layered on top of 150 mL of 85% ethanol antisolvent there were no HSA crystals formed. Accordingly, as numerous crystals were formed in other scenarios, the stirring was readded to the protocol. In the following experiment, the 10 mL of 0.9 M sodium fluoride salt solution was added to the 150 mL of 85% ethanol while stirring at 100 RPM. 88% of HSA crystals were formed in the precipitate.

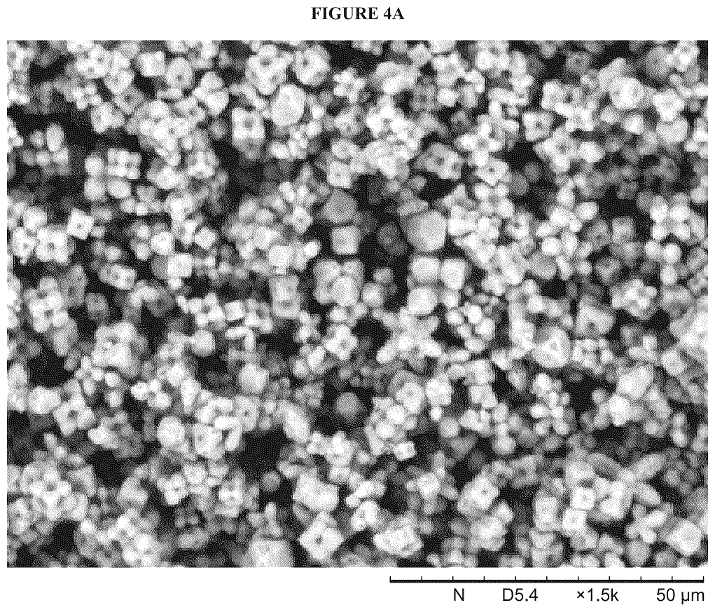

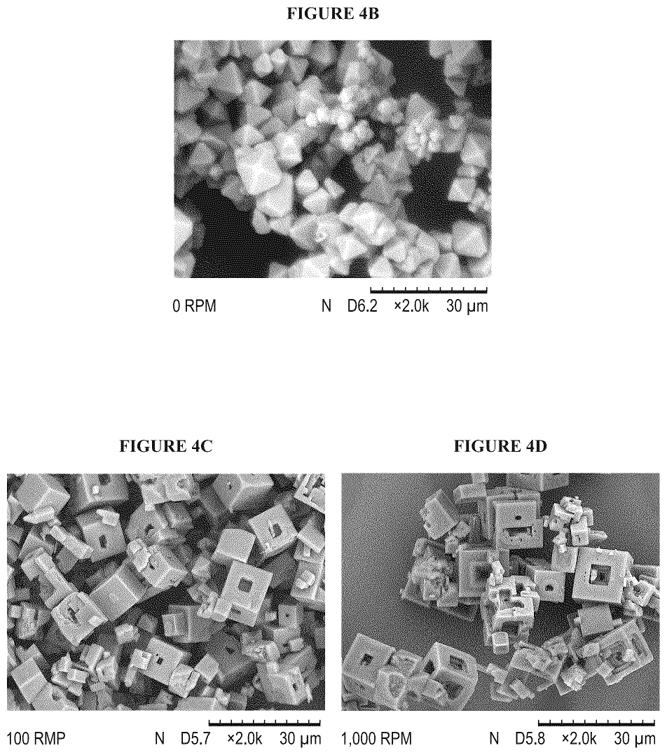

In the following experiment, the 10 mL of 0.9 M sodium fluoride salt solution was added to the 150 mL of 85% ethanol while stirring at 1,000 RPM. By increasing the stir rate to 1,000 RPM, the percent of HSA crystal formation was mildly increased to 90%, with the numbers being within an error bar of one another. The image, as depicted in FIG. 4A is representative of both the 100 RPM and 1,000 RPM stirring protocols. Indeed, even for certain other antisolvents, increasing the stir rate from 100 RPM to 1,000 RPM was necessary to generate any HSA crystal formation.

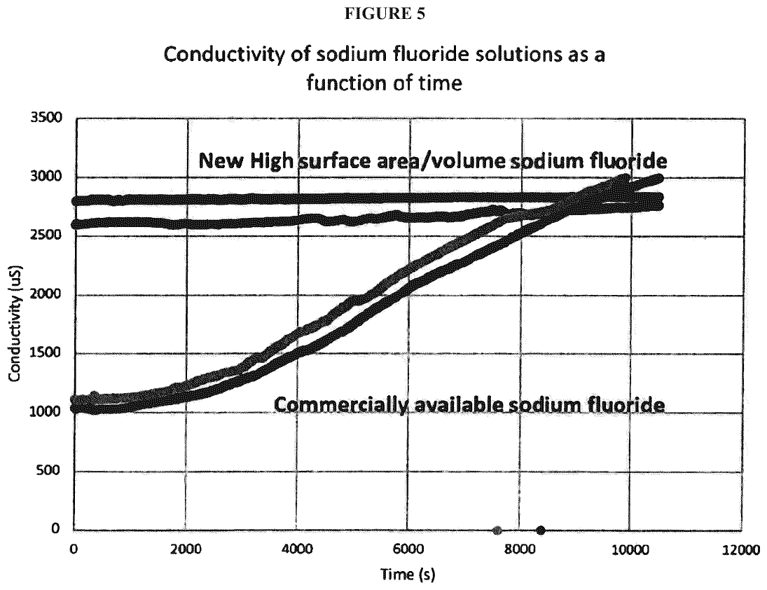

The results were repeated using the following parameters: 0.9 M NaF, added to antisolvent (85% ethanol) with a 5:1 ratio of antisolvent:NaF solution. The tests were again performed first at zero stirring, then at 100 RPM, and finally at 1,000 RPM. The results of this experiment are depicted in FIGS. 5B, 5C, and 5D. Notably, in the absence of stirring, HSA crystals are not formed. But by simply stirring at 100 RPM or 1,000 RPM, significant formation of HSA crystals is found.

Therefore, stirring of the antisolvent solution greatly increases the yield of HSA crystal formation. Using certain antisolvents, only upon increasing the stirring rate was it even possible to generate HSA crystals. Thus, while the precision of 100 RPM or 1,000 RPM is easy to quantify in a lab situation with an experimental size reaction, in a commercial application for formation of the HSA NaF crystals any form of stirring of the solution appears to be sufficient to increase the HSA crystal yield. When stirring is desired in a commercial scale, numerous methods can be used to replicate the mixing, which appears to increase the yield of HSA crystals. Thus, agitation or stirring, whether a large stir bar, aeration, or circulation pumps, as nonlimiting examples, can be utilized. Thus, it is preferable that stirring, mixing, or agitation of the solution will increase the yield. Accordingly, those of skill in the art will recognize that the novel sodium fluoride HSA crystals can be formed in small or commercial size quantities based upon the relative concentrations as detailed herein.

In summary, several variables are critical in the formation of HSA crystals. The choice of antisolvent and the degree to which it is diluted with water has a significant impact on formation of the desired product. Additionally, the ratio of salt solution to antisolvent and stirring of the mixture to increase yield are of great importance in the production of the targeted material. Indeed, the formation of the HSA NaF crystals depends on using the proper amount of the antisolvent, the ratio of salt to antisolvent and at least some stirring of the mixture when adding the salt solution to the antisolvent, and unexpected improvements in the yield were found by the particularly defined ratios of antisolvent to salt, as well as the antisolvent used, while stirring the materials during their combination.

### EXAMPLES

The synthesis of hopper-shaped crystals was tested through use of an antisolvent crystallization method. Several different variables were tested with series of experiments in order to find which combinations yielded crystals with a hopper morphology. 1 L of a 0.9 M sodium fluoride solution was prepared and used for all the following experiments. The antisolvent selected for each formulation was measured and transferred to a beaker. At room temperature, 5 mL of the NaF solution was either layered on top or dripped in the antisolvent. A stir bar was included in the beaker containing the antisolvent prior to adding the NaF, depending on the stir rate variable being tested. The beaker containing the NaF-antisolvent solution was labeled, covered with Parafilm, and either left on the lab bench or placed on a stir plate at a set stir rate for 24 hours.

After 24 hours, the beakers were uncovered and the top layer of remaining antisolvent was pipetted out and discarded. The solution was left uncovered to dry at room temperature for 24 hours. A spatula was then used to transfer the crystallized sample to a round-bottomed flask and placed on a vacuum dryer to evaporate any remaining solution. The flask containing the sample was placed in a silicone oil bath set at 100° C. for 48 hours. Then, the dried crystals were removed and transferred to a labeled vial.

Table 3, below, provides a relative summary of the results of several of the tests, and outlines that within certain reaction parameters, significant formation of the HSA crystals are formed, and outside of those conditions, the percent of HSA crystal formation approaches and reaches zero. Notably, an antisolvent percent (as a percentage of ethanol) of at between 85% and 90% appears to be surprisingly effective in creating high yields of HSA NaF crystals, when combined with a radio of antisolvent:NaF solution of at least 5:1 and preferably 10:1 or higher, such as to 15:1, when the solution is stirred or agitated at at least 100 RPM. Through these combinations, an unexpected crystal morphology at unexpectedly high yields was obtained.

TABLE 3

| | | Ratio of | | |
|---|---|---|---|---|
| | Antisolvent | Antisolvent:NaF | Stirrer Speed | % Hopper |
| Formulation | (% Ethanol) | solution | (RPM) | Crystals |
| 1 | 100 | 10:1 | 1,000 | 0 |
| 2 | 95 | 10:1 | 1,000 | 5 ± 2 |
| 3 | 90 | 10:1 | 1,000 | 85 ± 5 |
| 4 | 85 | 10:1 | 1,000 | 85 ± 5 |
| 5 | 80 | 10:1 | 1,000 | 2 ± 1 |
| 6 | 75 | 10:1 | 1,000 | 0 |
| 7 | 90 | 1.25:1 | 1,000 | 0 |
| 8 | 90 | 5:1 | 1,000 | 80 ± 5 |
| 9 | 90 | 15:1 | 1,000 | 85 ± 5 |
| 10 | 85 | 5:1 | 0 | 0 |
| 11 | 85 | 5:1 | 100 | 85 ± 5 |
| 12 | 85 | 5:1 | 1,000 | 85 ± 5 |

Additional studies were tried with 100% acetone, and 100% acetonitrile as the antisolvent. Subsequent tests were then performed using the above protocol with different concentrations of the ethanol and water, as well as modify the ratio of antisolvent to the NaF solution.

To demonstrate that the new HSA sodium fluoride crystals dissolve faster than commercially available sodium fluoride, the conductivity of the solution was measured as a function of time. Such a test is a standard test that is well understood in the scientific community. In this experiment, at room temperature, 35 mg of commercially available solid cubic sodium fluoride was added to a beaker. A conductivity probe was placed into the beaker. 25 mL of ultrapure water was added to the beaker, instantaneously submerging the conductivity probe. Conductivity data can be collected every 3 seconds on the probe. The peak conductivity of a sodium fluoride solution with this concentration is approximately 3,000 μS/cm. In the first 3 seconds, approximately 33% of the commercially available solid cubic sodium fluoride dissolves. The remaining sodium fluoride dissolves over the next 200 seconds to 800 seconds.

Contrast that to the HSA crystal NaF wherein approximately 90% of the salt dissolved within 3 seconds. The results of this test are depicted in the graph of FIG. 5. In order to characterize the structural differences between the commercial, solid cubic NaF and the claimed HSA crystal of the present disclosure, images were taken via scanning electron microscopy to show the visual differences. However, to further characterize the HSA NaF crystals, the Hall density of the hopper crystals of sodium fluoride was compared to commercial solid cubic crystals of sodium fluoride (ASTM Standard B212-99). Commercial solid cubic crystals of sodium fluoride had a Hall density of 0.86±0.03 g/cm³. By contrast, the HSA NaF hopper crystals resulted in a Hall density of 0.68±0.04 g/cm³. This provides an easy way to identify the differences in the materials.

Further characterization of the HSA NaF crystals compared to the solid cubic commercially available crystals of sodium fluoride examined the surface area (BET method) normalized to the mass of the crystals. The commercial solid cubic crystals had a significantly lower surface area to mass ratio than the hopper crystals. The commercial crystals were 0.12±0.02 m²/g whereas the hopper crystals exhibited a significantly higher surface area of 0.22±0.04 m²/g. Use of either of the Hall density of the surface area normalized to mass allows for a quantitative mechanism to differentiate the crystal formation.

Table 4 further details some points for dissolution of the salt at various times.

TABLE 4

| | 3 sec | 200 sec | 400 sec | 800 sec |
|---|---|---|---|---|
| Salt Type | Dissolution | Dissolution | Dissolution | Dissolution |
| Ordinary | ~1,000 | ~1,175 | ~1,500 | ~2,500 |
| High Surface | ~2,600 | ~2,625 | ~2,700 | ~2,750 |
| Area | | | | |

The data in Table 4 is significant to a number of issues in the oral care industry. First, the data points at 3 seconds and then at 200 seconds shows the slow progression of the dissolution of the ordinary salt over a period of more than 3 minutes of time. As the ordinary user only brushes for as little as a few seconds to usually 2 minutes or less, very little additional fluoride has dissolved beyond the initial dissolution. Thus, while it takes the HSA crystal only 3 seconds to reach virtual maximum release, it is more than ten minutes into the dissolution for the ordinary solid cubic crystal to reach this concentration. Thus, the HSA crystals show a significant advantage in their rate of dissolution in water, which is advantageous when typical use of sodium fluoride is a time of 2 minutes or less.

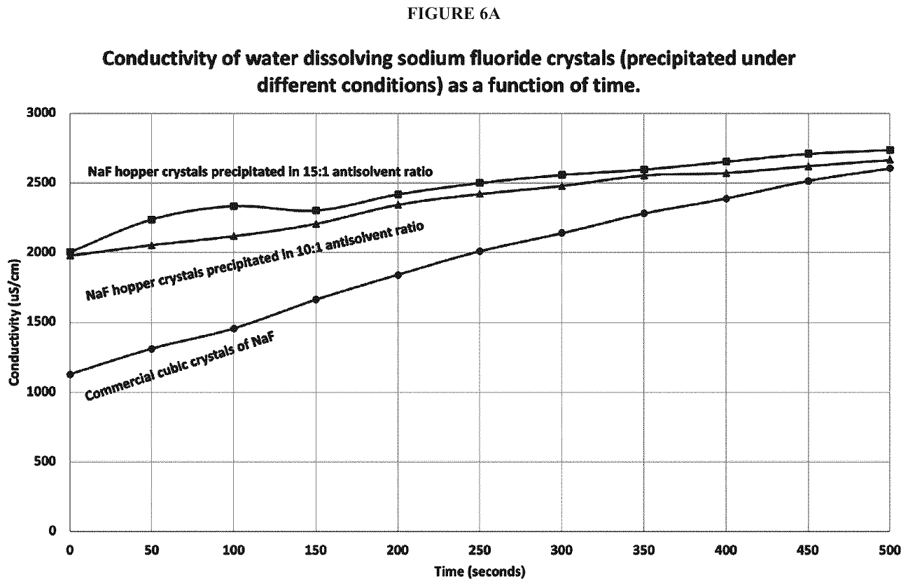

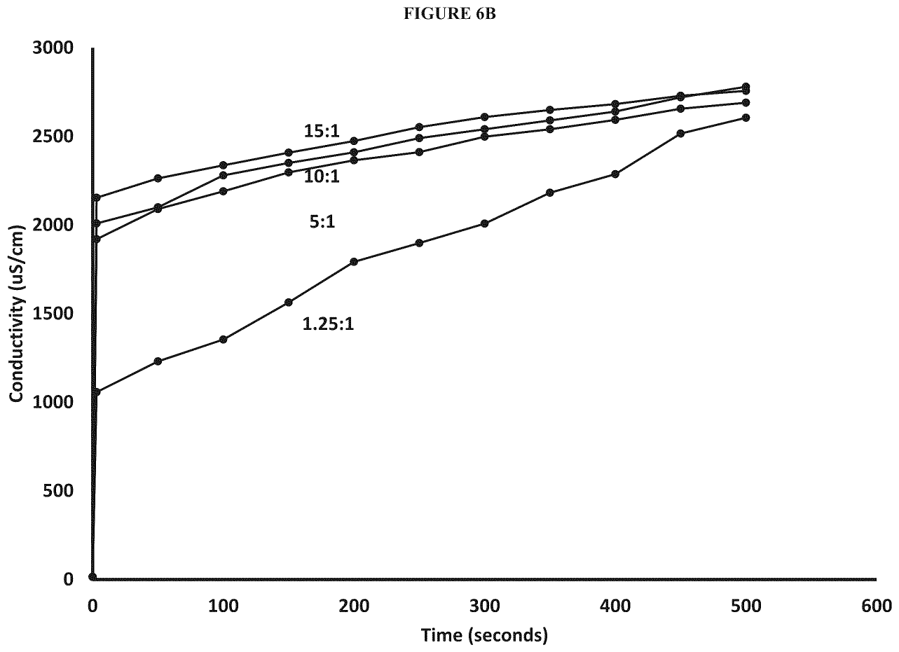

FIGS. 6A, 6B, 7A, 7B, 8A, and 8B provide some further examples of the dissolution rates of the high surface area crystals under different protocols as outlined above, as compared to the control of the ordinary solid cubic crystals. FIG. 6A, for example depicts a comparison of the conductivity of the HSA crystals formed at antisolvent to salt solution ratios of 10:1 and 15:1. This is tested by adding to a beaker 56 mg of the given salt to be tested. To this is added a conductivity probe. As the salt is added, the solution is stirred. FIG. 6B shows a further test, comparing the crystals formed at four different ratios of antisolvent:NaF, ranging from 1.25:1 to 15:1, when run with 90% ethanol and stirring at 1,000 RPM.

Advantageously, where ordinary solid cubic NaF salt crystals are utilized, these can be replaced by the HSA crystals of the present disclosure, which provide a distinctive advantage over the prior art crystals based on a higher dissolution rate of the HSA NaF crystal. Preferably, these materials are added into oral care products.

The experiments detail herein and as provided by this disclosure provide for methods of forming high yields of HSA NaF crystals that are shown to dissolve faster in the oral environment than ordinary cubic NaF crystals. This could lead to a significant improvement in preventing and treating dental caries and periodontal disease among other treatments. The antisolvent approach to precipitation was implemented in these experiments to induce growth of hopper-shaped crystals. As it is best understood, though the mechanism described here does not limit the process, product, or compositions using the HSA NaF crystals, a concentration gradient forms around a growing cubic crystal in a saturated or near saturated solution, with the solute concentration being greater at the corners and edges than at the faces. The growth of a cubic crystal is governed by kinetics. Addition of an antisolvent creates a supersaturated solution, amplifying the effects of the concentration gradient. Under the right conditions, growth can then become diffusion limited, leading to the formation of hopper crystals.

The use of alcohol plays two major roles in the growth of hopper crystals, including viscosity and solubility. First, the viscosity of the antisolvent solution system increases as the water concentration purposefully added to the antisolvent increases from 1 w/w % to 15 w/w %. This is evidenced by the higher yields of hopper crystals in experiments with 10:1 and 15:1 ratios. As the solution becomes more viscous, this inhibits the diffusion of ions to the crystal structure, potentially favoring the formation of a hopper crystal. Second, the solubility of NaF in solution is decreased as alcohol concentration increases. As the solubility of NaF decreases, the growth rate of the crystal increases. An increased growth rate leads to a greater concentration gradient at the interface of the growing crystal, potentially promoting hopper crystal growth.

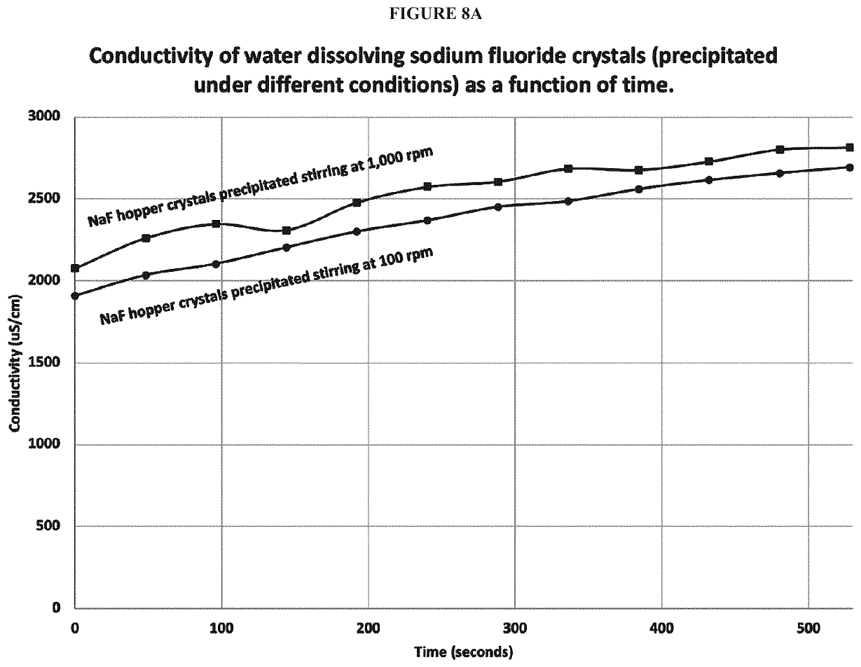

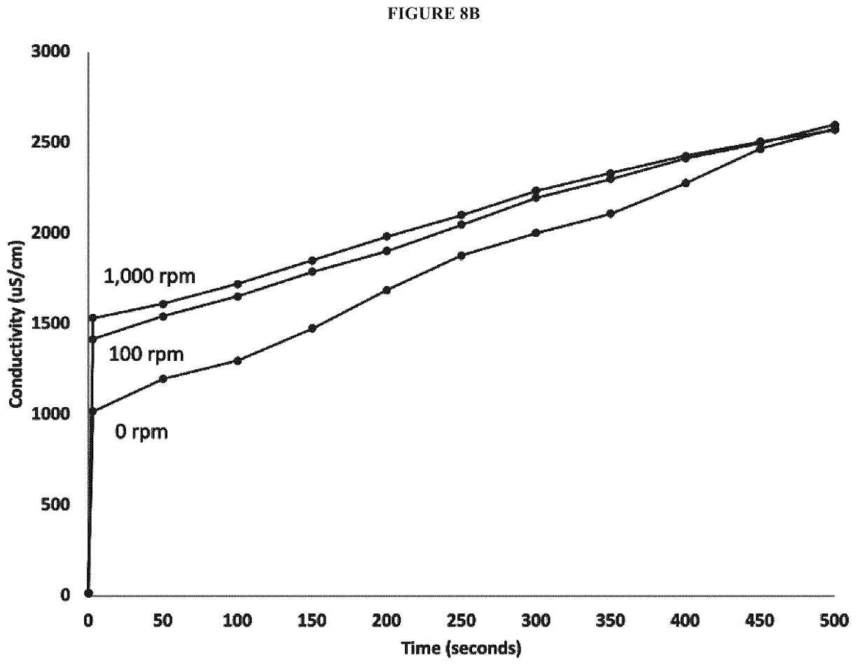

The dissolution rate of NaF crystals correlates to the conductivity of NaF in solution. That is to say, as salt dissolves in water, the conductivity of the salt solution increases. FIG. 7A depicts the dissolution of three different sources of NaF crystals. Conductivity is utilized to test the rate of dissolution. The conductivity of ultrapure water is approximately 14 μS/cm when no ions are present. The first reading on the conductivity meter can be made 3 seconds after the experiment begins. Initial reading on the conductivity meter for the commercial NaF was 1,127 μS/cm. After all of the salt dissolved, the conductivity was 2,605 μS/cm. This suggests that ˜43% of the NaF was dissolved in the opening 3 seconds. About 500 seconds passed before all the NaF dissolved. Contrast this to the two different dissolution plots for the HSA crystals prepared where the ratio of antisolvent to NaF was 10:1 and stirred at 100 RPM with either 85% ethanol or 90% ethanol as the antisolvent. In the case of the 85% ethanol formulation 2,068 μS/cm or approximately 79% of the crystals dissolved in the first 3 seconds. Similarly, the crystals made from precipitation into 90% ethanol resulted in 60% of the crystals dissolving in the first 3 seconds. FIG. 7B then depicts a second chart, depicting conductivity at four different antisolvent compositions, namely at 80%, 85%, 90%, and 95% ethanol, each with a ratio of 10:1 antisolvent to salt, and stirred at 1,000 RPM. This data suggests that the percentage of ethanol as the antisolvent affects the final distribution of hopper morphology and the effective surface area to volume ratio of the crystals. In addition to the percentage of ethanol in the antisolvent, the ratio of antisolvent to NaF solution (FIGS. 6A and 6B) and stir rate (FIGS. 8A and 8B) affects the final surface area to volume ratio of the NaF crystals prepared.

FIG. 8A shows that both the 100 RPM and 1,000 RPM conductivity is similar, while FIG. 8B depicts that the lack of stirring, even when created with optimized conditions of 85% ethanol and an antisolvent to Naf ratio of 5:1, yields crystal formation that replicates commercial crystals.

The hypothesis of this research is that NaF crystals with larger surface area to volume ratios can dissolve faster in solution, which is borne out in these studies. Syntheses that resulted in hopper shaped crystals highlighted three key variables in the formation of these morphologies. These variables include the concentration of the antisolvent, the ratio of antisolvent to NaF solution, and the stir rate. Therefore, the conductivity of hopper crystals of NaF were measured as a function of time varying these conditions and compared against cubic crystals of commercial grade NaF. Hopper crystals of NaF were dissolved in an aqueous environment at a greater rate than commercially available cubic crystals. This suggests that the inclusion of NaF hopper crystals in fluoride treatments could improve remineralization of enamel.

### Example 1

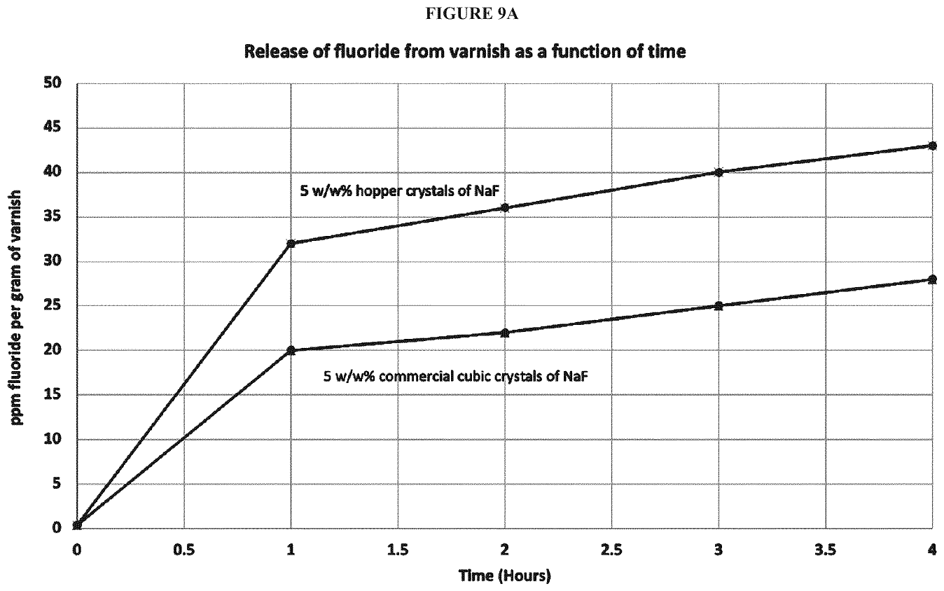

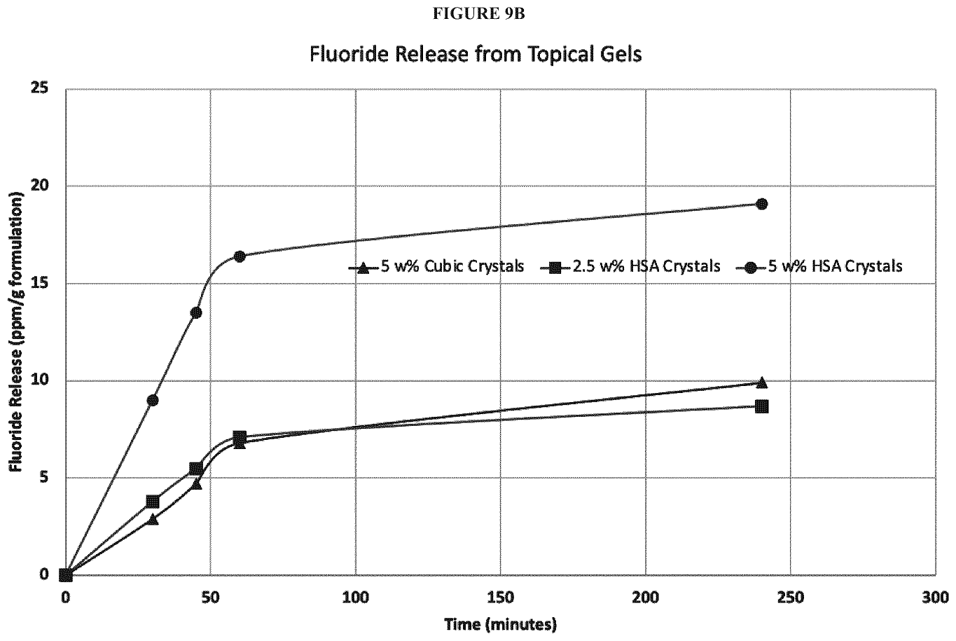

A varnish with HSA crystals of sodium fluoride was prepared. A mixing cup was filled with 3 grams of rosin and 1 gram of ethanol. The components were mixed until the blend was homogenous. 0.2 grams of sodium fluoride HSA crystals were added to the mixture. The components were mixed until the HSA sodium fluoride crystals were dispersed in the rosin:ethanol blend. This product was tested and compared against a commercially available sodium fluoride varnish in FIG. 9A.

### Example 2

A dentifrice with HSA crystals of sodium fluoride was prepared. 30 grams of glycerin, 1.5 grams of sodium lauryl sulphate, 35 grams of silica, 1 gram of methyl cellulose, 0.3 grams of sodium saccharine, 0.1 gram of methyl paraben, 0.5 grams of titanium dioxide, and 1.5 grams of menthol were homogenized. 1,150 ppm of HSA sodium fluoride was mixed into the homogenized blend.

Finally, FIG. 9A details a sample of a commercial product utilizing 5 w/w % of commercial cubic crystals of NaF compared to a varnish using 5 w/w % of the HSA crystals of NaF as detailed herein. The varnish was prepared by mixing rosin and ethanol in a 3:1 ratio as provided above. HSA NaF crystals, as prepared by the protocols in this disclosure were added to the rosin:ethanol blend to make the concentration 5 w/w % sodium fluoride.

The results of the comparison are striking in the release profile of the two materials. The material using the HSA NaF crystals of the present disclosure releases fluoride significantly faster than the solid cubic commercial NaF crystal-based material. As the varnish is often used for only a few hours' time, before it is removed from the tooth and oral mucosa by saliva and tongue action, note that the dissolution of the solid cubic commercial crystal reaches only approximately 28 ppm at 4 hours, while the HSA crystal is above 32 ppm at 1 hour. Thus, the HSA crystal provides a significant advantage in release in the varnish product as compared to the solid cubic prior art crystals. Thus, it may be possible to reduce the w/w % of the HSA crystals in the materials and obtain the same therapeutic effect as compared to the ordinary cubic crystals. Indeed, FIG. 9B depicts just this example, by reducing the amount of the HSA crystal example to 2.5% w/w %. Notably, this material performs in a similar manner to that of the commercial crystals at 5%, though both are significantly inferior as compared to the 5% w/w % HSA crystal example. The 2.5% material releases at least 5 ppm/g formulation of fluoride in 50 minutes or less, which is higher than the commercial formulation using solid commercial NaF crystals. Thus, the HSA crystal could easily be used at ½ of the concentration of the commercial crystal and obtain a similar fluoride release over time. However, the time here is in minutes, and, as was previously shown, the HSA crystal has a much faster rate of dissolution, and thus an even lower concentration of HSA crystals would likely be sufficient to obtain a similar fluoride release within the first 1 minute, which would be analogous to the normal brushing time under ordinary use.

Indeed, as nonlimiting examples, the rapid, burst like release of fluoride from the high surface area crystals will be advantageous to numerous oral care products such as oral toothpastes and gels, desensitization adjunct in tooth whitening gels, desensitization gel for sensitive teeth and periodontal postoperative treatment, dental varnish, nonaqueous dentifrices, glass ionomer restorative materials and cements, resin modified glass ionomer materials and cements, resin restorative dental materials, flowables, pastes, cements, dental adhesives, glazes, and endodontic sealers, flosses and other interproximal devices.

Those of ordinary skill in the art will recognize that the unique crystal morphology will yield rapid dissolution of the fluoride ion and will be advantageous in a wide variety of oral care products.

What is claimed is:

1. A method of manufacture of a high surface area (HSA) sodium fluoride (NaF) crystal comprising:
a. dissolving a portion of NaF in water to form an NaF solution;
b. forming an antisolvent solution; and
c. adding the NaF solution into the antisolvent solution to precipitate NaF crystals out of the NaF solution.

2. The method of claim 1 further comprising stirring the antisolvent solution while adding the NaF solution.

3. The method of claim 1 wherein the NaF solution is between 0.8 M and 0.95 M NaF.

4. The method of claim 1 wherein the antisolvent solution comprises an impurity of water.

5. The method of claim 1 wherein the antisolvent solution is ethanol and water.

6. The method of claim 1 wherein the antisolvent solution is between 80% and 95% ethanol.

7. The method of claim 1 wherein the ratio of antisolvent solution to NaF solution is 5:1 to 15:1.

8. The method of claim 7 wherein the ratio of antisolvent solution to NaF solution is 10:1 to 15:1.

9. The method of claim 1 further comprising a drying step comprising removing the antisolvent solution.

10. The method of claim 1 further comprising mixing the NaF and antisolvent solution for a predetermined amount of time to allow for HSA crystal formation and upon expiration of the predetermined amount of time removing a layer of antisolvent; and wherein the predetermined amount of time is between 1 hour and 24 hours.

11. The method of claim 1 further comprising one or more of the following steps:
d. drying the HSA crystals and solution at room temperature;
e. vacuum drying the HSA crystals; and
f. placing a container containing formed crystals into an oil bath at 100° C.

12. (canceled)

13. The method of claim 1 wherein the HSA NaF salt crystals are defined by a Hall density of 0.68±0.04 g/cm³ and/or a surface area to mass ratio of 0.22±0.04 m²/g.

14. (canceled)

15. (canceled)

16. An oral care product comprising a nonaqueous carrier and a portion of sodium fluoride hopper crystals.

17. (canceled)

18. The oral care product of claim 16 wherein the sodium fluoride hopper crystal comprises a Hall density of 0.68±0.04 g/cm³ and/or a surface area to mass ratio of 0.22±0.04 m²/g.

19. The oral care product of claim 16 wherein the sodium fluoride hopper crystals are provided in the oral care product at 2.5 w/w %.

20. (canceled)

21. (canceled)

22. (canceled)

23. A sodium fluoride crystal having a Hall density of 0.68±0.04 g/cm³ and/or a surface area to mass ratio of 0.22±0.04 m²/g which is made by a method comprising the following steps:
a. dissolving a portion of NaF in water to form an NaF solution of at least 0.8 M;
b. forming an antisolvent solution comprised of ethanol and water with ethanol at between 80% and 95%; and
c. adding the NaF solution into the antisolvent solution to precipitate NaF crystals out of the NaF solution while agitating the antisolvent solution.

24. The oral care product of claim 16 wherein the sodium fluoride hopper crystal is present in a sufficient quantity and structure to provide a release profile of at least 50 ppm/g formulation within 50 minutes.
